(19) United States
(12) Patent Application Publication
(10) Pub. No.: US 2025/0082456 A1
(43) Pub. Date: Mar. 13, 2025

(54) STENT WITH DYNAMIC ANTI-MIGRATION PROPERTIES

(71) Applicant: BOSTON SCIENTIFIC SCIMED, INC., Maple Grove, MN (US)

(72) Inventor: MARTYN G. FOLAN, Galway (IE)

(73) Assignee: BOSTON SCIENTIFIC SCIMED, INC., Maple Grove, MN (US)

(21) Appl. No.: 18/826,920

(22) Filed: Sep. 6, 2024

Related U.S. Application Data
(60) Provisional application No. 63/581,074, filed on Sep. 7, 2023.

(51) Int. Cl. A61F 2/04

(52) U.S. Cl. CPC A61F 2/04; A61F 2002/044; A61F 2230/0006; A61F 2230/006; A61F 2250/0037; A61F 2250/0039

(57) ABSTRACT

A medical stent includes an elongate tubular body extending from a proximal end region to a distal end region. The distal end region of the elongate tubular body may be adapted to resist migration as a result of peristaltic force. The distal end region of the elongate tubular body may include two or more longitudinally-extending voids and two or more longitudinally-extending dynamic engagement features that are defined between adjacent longitudinally-extending voids. The two or more longitudinally-extending dynamic engagement features are adapted to splay radially outwardly in response to an applied peristaltic force, thereby dissipating the applied peristaltic force.

### CROSS REFERENCE TO RELATED APPLICATIONS

The present application claims the benefit of and priority to U.S. Provisional Patent Application Ser. No. 63/581,074, filed on Sep. 7, 2023, the disclosure of which is incorporated herein by reference.

### TECHNICAL FIELD

The present disclosure relates generally to methods and apparatuses for various ailments. More particularly, the disclosure relates to different configurations and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain patency of the structure. These devices are manufactured by any one of a variety of different manufacturing methods and may be used according to any one of a variety of methods for a variety of applications. Of the known medical stents, delivery systems, and methods, each has certain advantages and disadvantages. For example, in some stents, the compressible and flexible properties that assist in stent delivery may also result in a stent that has a tendency to migrate from its originally deployed position in a body lumen. As an example, stents that are designed to be positioned in the esophageal or gastrointestinal tract may have a tendency to migrate due to peristalsis (i.e., the involuntary constriction and relaxation of the muscles of the esophagus, intestine, and colon which push the contents of the canal therethrough). There is an ongoing need to provide alternative medical stents and delivery devices as well as alternative methods for manufacturing and using medical stents and delivery devices, such as those susceptible to migration in the anatomy.

### SUMMARY

The disclosure is directed to several alternative designs, materials and methods of manufacturing medical device structures and assemblies, and the use thereof. An example may be found in a medical device, such as a stent, including an elongate tubular body extending from a proximal end region to a distal end region. The distal end region of the elongate tubular body is adapted to resist motion as a result of peristaltic force and includes two or more longitudinally-extending voids and two or more longitudinally-extending dynamic engagement features that are defined between adjacent longitudinally-extending voids. The two or more longitudinally-extending dynamic engagement features are adapted to splay radially outwardly in response to an applied peristaltic force, thereby dissipating the applied peristaltic force.

Alternatively or additionally, the distal end region extends to a distal end of the elongate tubular body, and the two or more longitudinally-extending voids may extend distally to the distal end.

Alternatively or additionally, the medical stent may have an overall length, and a length of each of the two or more longitudinally-extending voids may be 25 percent or less of the overall length.

Alternatively or additionally, the elongate tubular body may have a constant diameter from the proximal end region to the distal end region when the elongate tubular body is in a relaxed state.

Alternatively or additionally, the distal end region of the elongate tubular body may have an increased diameter relative to a diameter of the elongate tubular body proximal of the distal end region.

Alternatively or additionally, the proximal end region of the elongate tubular body may have an increased diameter relative to a diameter of the elongate tubular body distal of the proximal end region.

Alternatively or additionally, the medical stent may further include a bulbous region disposed between the distal end region and the proximal end region.

Alternatively or additionally, the medical stent may further include a polymeric coating extending over at least a portion of the elongate tubular body.

Alternatively or additionally, the polymeric coating may extend from the proximal end region to the distal end region.

Alternatively or additionally, the two or more longitudinally-extending voids are devoid of the polymeric coating.

Alternatively or additionally, the two or more longitudinally-extending dynamic engagement features may be adapted to be atraumatic.

Another example may be found in a medical device, such as a medical stent. The medical stent includes an elongate tubular body extending from a proximal end region to a distal end region and a polymeric coating extending over at least a portion of the elongate tubular body. At least one of the proximal end region and the distal end region is adapted to resist migration in a body lumen as a result of an applied force, the region configured to resist migration including a plurality of longitudinally-extending voids and a plurality of longitudinally-extending dynamic engagement features defined between adjacent longitudinally-extending voids. The plurality of longitudinally-extending dynamic engagement features are adapted to splay radially outwardly in response to the applied force, thereby dissipating the applied force.

Alternatively or additionally, the region configured to resist migration as a result of the applied force may include the distal end region.

Alternatively or additionally, the region configured to resist migration as a result of the applied force may include the proximal end region.

Another example may be found in a medical device, such as a medical stent. The medical stent includes an elongate tubular body extending from a proximal end region to a distal end region, the elongate tubular body including two or more longitudinally-extending voids extend proximally from a distal end of the elongate tubular body and defining two or more two or more longitudinally-extending dynamic engagement features therebetween. The two or more longitudinally-extending dynamic engagement features are adapted to independently splay radially outwardly in response to an applied peristaltic force, thereby dissipating the applied peristaltic force.

Another example may be found in a medical device, such as a medical stent. The medical stent includes an elongate tubular body extending from a proximal end region to a distal end region. The distal end region of the elongate tubular body is adapted to resist motion as a result of peristaltic force and includes two or more longitudinally-extending voids and two or more longitudinally-extending dynamic engagement features that are defined between adjacent longitudinally-extending voids. The two or more longitudinally-extending dynamic engagement features are adapted to splay radially outwardly in response to an applied peristaltic force, thereby dissipating the applied peristaltic force.

Alternatively or additionally, the distal end region extends to a distal end of the elongate tubular body, and the two or more longitudinally-extending voids extend distally to the distal end.

Alternatively or additionally, each of the two or more longitudinally-extending voids may have the same length.

Alternatively or additionally, at least some of the two or more longitudinally-extending voids may have a different length.

Alternatively or additionally, the medical stent may have an overall length, and the length of each of the two or more longitudinally-extending voids may be 25 percent or less of the overall length.

Alternatively or additionally, the elongate tubular body may have a constant diameter from the proximal end region to the distal end region when the elongate tubular body is in a relaxed state.

Alternatively or additionally, the distal end region of the elongate tubular body may have an increased diameter relative to a diameter of the elongate tubular body proximal of the distal end region.

Alternatively or additionally, the proximal end region of the elongate tubular body may have an increased diameter relative to a diameter of the elongate tubular body distal of the proximal end region.

Alternatively or additionally, the elongate tubular body may include a braided stent body.

Alternatively or additionally, the elongate tubular body may include a knitted stent body.

Alternatively or additionally, the medical stent may further include a bulbous region disposed between the distal end region and the proximal end region.

Alternatively or additionally, the medical stent may further include a polymeric coating extending over at least a portion of the elongate tubular body.

Alternatively or additionally, the polymeric coating may extend from the proximal end region to the distal end region.

Alternatively or additionally, the two or more longitudinally-extending voids are devoid of the polymeric coating.

Alternatively or additionally, the two or more longitudinally-extending dynamic engagement features may be adapted to be atraumatic.

Another example may be found in a medical device, such as a medical stent. The medical stent includes an elongate tubular body extending from a proximal end region to a distal end region and a polymeric coating extending over at least a portion of the elongate tubular body. At least one of the proximal end region and the distal end region is adapted to resist migration as a result of an applied force, the region configured to resist migration including a plurality of longitudinally-extending voids and a plurality of longitudinally-extending dynamic engagement features defined between adjacent longitudinally-extending voids. The plurality of longitudinally-extending dynamic engagement features are adapted to independently splay radially outwardly in response to the applied force, thereby dissipating the applied force.

Alternatively or additionally, the region configured to resist migration as a result of the applied force may include the distal end region.

Alternatively or additionally, the region configured to resist migration as a result of the applied force may include the proximal end region.

Another example may be found in a medical device, such as a medical stent. The medical stent includes an elongate tubular body extending from a proximal end region to a distal end region, the elongate tubular body including two or more longitudinally-extending voids extend proximally from a distal end of the elongate tubular body and defining two or more two or more longitudinally-extending dynamic engagement features therebetween. The two or more longitudinally-extending dynamic engagement features are adapted to independently splay radially outwardly in response to an applied peristaltic force, thereby dissipating the applied peristaltic force.

Alternatively or additionally, the medical stent may further include a polymeric covering extending over at least a portion of the elongate tubular body.

Alternatively or additionally, each of the two or more longitudinally-extending dynamic engagement features includes a plurality of filament cross-over points in which either 1) a first filament of the elongate tubular body crosses over a second filament of the elongate tubular body, or 2) a first filament segment of a filament of the elongate tubular body crosses over a second filament segment of the filament.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a schematic view of an example medical stent;

FIG. 2 is a schematic view of an example medical stent;

FIGS. 3A and 3B are schematic end views of an example medical stent;

FIGS. 4A and 4B demonstrate how an example medical stent responds to an applied axial force;

FIG. 5 is a schematic view of an example medical stent;

FIG. 6 is a schematic view of an example medical stent;

FIG. 7 is a schematic view of an example medical stent;

FIG. 8 is a schematic view of an example medical stent;

FIG. 9 is a schematic view of an example medical stent;

FIG. 10 is a schematic view of an example medical stent;

FIG. 11 is a schematic view of a portion of an example medical stent; and

FIG. 12 is a schematic view of a portion of an example medical stent.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

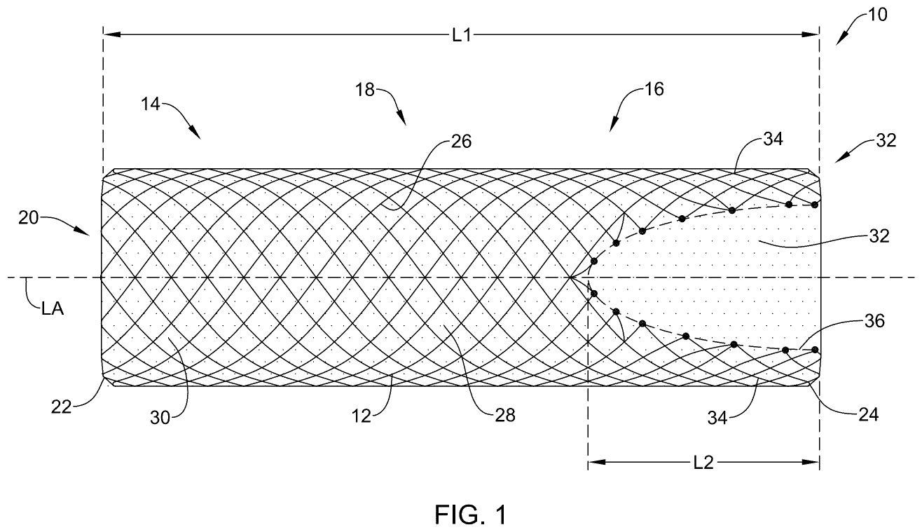

Stents are utilized in a variety of different body lumens, including the vasculature and various parts of the gastrointestinal system, for example. In some instances, particularly in gastrointestinal applications, an implanted stent may be subjected to a variety of different forces. For example, the gastrointestinal system may view the stent as a foreign object that should be expelled. In some instances, some parts of the gastrointestinal system may attempt to force an implanted stent to move in a distal direction. In some instances, some parts of the gastrointestinal system may attempt to force an implanted stent to move in a proximal, or upward and outward, direction. It will be appreciated that some parts of the gastrointestinal system naturally undergo movement such as peristaltic movement in order to cause food and food substances such as partially digested food to move distally through the gastrointestinal system. Stents placed in various parts of the gastrointestinal system, such as but not limited to the esophagus, are subjected to peristaltic movement. FIG. 1 is a schematic view of an example medical device, illustrated as a medical stent 10, that is adapted for use in body lumens in which the medical device will be subjected to various movement forces. The example medical device may be considered as being an endoluminal device such as a medical stent or endoprosthesis, for example.

In FIG. 1, the example medical stent 10 includes an elongate tubular body 12. While the medical stent 10 is described as generally tubular, it is contemplated that the medical stent 10 may take any cross-sectional shape desired. The elongate tubular body 12 may be considered as including a proximal end region 14, a distal end region 16, and an intervening intermediate region 18, recognizing that these designations are arbitrary, and depend on the orientation in which the medical stent 10 will ultimately be implanted within a body lumen. In some instances, the elongate tubular body 12 may be considered as having a constant diameter throughout, including through the proximal end region 14, the distal end region 16 and the intervening intermediate region 18. In some instances, the elongate tubular body 12 may be considered as having a constant diameter when in a relaxed state. In other instances, the proximal end region 14 and/or the distal end region 16 may include an enlarged diameter portion (e.g., flared region), if desired. The medical stent 10 may include a lumen 20 that extends from a proximal end 22 of the elongate tubular body 12 to a distal end 24 of the elongate tubular body 12 to allow for the passage of food, fluids, etc. The medical stent 10 may be considered as having a longitudinal axis LA extending axially through the lumen 20. The medical stent 10 may be expandable from a first radially collapsed configuration (not shown) to a second radially expanded configuration, which may correspond to its relaxed or equilibrium state. In some instances, the medical stent 10 may be structured to extend across a stricture and to apply a radially outward pressure to the stricture in a body lumen in order to open the body lumen and allow for the passage of foods, fluids, air, etc. therethrough.

In some instances, the medical stent 10 may have a woven structure, fabricated from a number of filaments or struts 26 that together form the elongate tubular body 12. In some instances, the medical stent 10 may be knitted or braided with a single filament or strut interwoven with itself and defining open cells 28 extending through a wall forming the elongate tubular body 12. In some instances, the medical stent 10 may be braided with several filaments or struts interwoven together and defining open cells 28 extending along a length and around the circumference of the tubular wall of the medical stent 10. The open cells 28 may each define an opening from an outer surface of the tubular wall to an inner surface of the tubular wall (e.g., through a thickness thereof) that is free from the filaments or struts 26. Some exemplary stents including braided filaments include the WallFlex®, WALLSTENT®, and Polyflex® stents, made and distributed by Boston Scientific, Corporation. In another embodiment, the medical stent 10 may be knitted, such as the Ultraflex™ stents made by Boston Scientific, Corporation. In yet another embodiment, the medical stent 10 may be of a knotted type, such the Precision Colonic™ stents made by Boston Scientific, Corporation. In still another embodiment, the medical stent 10 may be a monolithic tubular member, for example a laser cut tubular member, such as the EPIC™ stents made by Boston Scientific, Corporation. A laser cut tubular member may have an open and/or closed cell geometry including one or more interconnected monolithic filaments or struts defining open cells 28 therebetween, with the open cells 28 extending along a length and around the circumference of the tubular wall. The open cells 28 may each define an opening from an outer surface of the tubular wall to an inner surface of the tubular wall (e.g., through a thickness thereof) that is free from the interconnected monolithic filaments or struts. In some instances, an inner and/or outer surface of the elongate tubular body 12 may be entirely, substantially, or partially, covered with a polymeric coating 30 that is illustrated as a dotted pattern over the elongated tubular member 12. The polymeric coating 30 may extend across and/or occlude one or more, or a plurality of the open cells 28 defined by the struts or filaments 26. The polymeric coating 30 may help reduce food impaction and/or tumor or tissue ingrowth. In some cases, the medical stent 10 may be a self-expanding stent (SES), configured to automatically radially expand when unconstrained, although this is not required.

In some instances, the elongate tubular body 12 of the medical stent 10 may be made from a number of different materials such as, but not limited to, metals, metal alloys, shape memory alloys and/or polymers, as desired, enabling the medical stent 10 to be expanded into shape when accurately positioned within the body. In some instances, the material may be selected to enable the medical stent 10 to be removed with relative ease as well. For example, the elongate tubular body 12 of the medical stent 10 can be formed from alloys such as, but not limited to, nitinol and Elgiloy®. Depending on the material selected for construction, the medical stent 10 may be self-expanding or require an external force to expand the medical stent 10. In some embodiments, composite filaments may be used to make the medical stent 10, which may include, for example, an outer shell or cladding made of nitinol and a core formed of platinum or other radiopaque material. It is further contemplated the elongate tubular body 12 of the medical stent 10 may be formed from polymers including, but not limited to, polyethylene terephthalate (PET). In some instances, the filaments of the medical stent 10, or portions thereof, may be bioabsorbable or biodegradable, while in other instances the filaments of the medical stent 10, or portions thereof, may be biostable.

In some instances, the medical stent 10 may be adapted to resist applied forces such as but not limited to those caused by peristaltic motion. In some instances, part of the medical stent 10, such as the distal end region 16 of the medical stent 10, may be adapted to resist motion that would otherwise tend to cause the medical stent 10 to migrate in response to an applied force such as that applied during peristaltic movement. In some instances, the distal end region 16 may include a plurality of longitudinally-extending voids 32, such as two or more longitudinally-extending voids 32, although only a single longitudinally-extending void 32 is visible in this view. Each of the two or more longitudinally-extending voids 32 may be considered as extending from a terminal end (e.g., the distal end 24) in a direction parallel to, or at least substantially parallel to, the longitudinal axis LA. Substantially parallel may be defined as within ten percent of parallel, for example. In some instances, while not shown, one or more of the longitudinally-extending voids 32 may not be parallel or substantially parallel to the longitudinal axis LA, but may instead be disposed at an acute angle with respect to the longitudinal axis LA. In some instances, the distal end region 16 may include a second longitudinally-extending void 32 that is circumferentially spaced about 180 degrees about the elongate tubular body 12. The distal end region 16 may include two, three, four or more longitudinally-extending voids 32, for example.

The longitudinally-extending voids 32 may open out to the distal extent of the medical stent 10 at the distal circumferential edge of the tubular body 12. In some instances, the two or more longitudinally-extending voids 32 may be considered as dividing the distal end region 16 into two or more longitudinally-extending dynamic engagement features 34, e.g., longitudinally-extending legs defined by portions of the wall of the tubular body 12. The two or more longitudinally-extending voids 32 may be considered as defining the longitudinally-extending dynamic engagement features 34 between circumferentially adjacent longitudinally-extending voids 32. In some instances, the two or more longitudinally-extending dynamic engagement features 34 may be considered as being adapted to independently splay radially outwardly in response to an applied peristaltic force, thereby dissipating the applied peristaltic force. In some instances, each of the two or more longitudinally-extending voids 32 extend distally to the distal end 24 of the elongate tubular body 12, such that the distal circumferential edge of the medical stent 10 includes discontinuous segments of the tubular body 12. In some instances, each of the two or more longitudinally-extending voids 32 have the same longitudinal length. In some instances, at least some of the two or more longitudinally-extending voids 32 may have a different length, resulting in varying lengths for the longitudinally-extending dynamic engagement features 34 that are defined at least in part by the two or more longitudinally-extending voids 32.

The elongate tubular body 12 may be considered as having an overall length L₁ that defines an overall length of the medical stent 10. It will be appreciated that the overall length L₁ may vary depending on the ultimate use of the medical stent 10. For an esophageal application, the length L₁ may vary from about 40 millimeters to about 200 millimeters. For a biliary application, the length L₁ may vary from about 40 millimeters to about 140 millimeters. These dimensions are exemplary. Each of the two or more longitudinally-extending voids 32 may be considered as having a length L₂. In some instances, the length L₂ may range from 5 millimeters to 80 millimeters. In some instances, the length L₂ may range from about 5 percent of the length L₁ to about 40 percent of the length L₁. In some instances, there is a desire to center the medical stent 10 on a stricture or treatment area, with equal portions of the medical stent 10 on either side of the stricture or treatment area. In some instances, the length L₂ is selected to permit the elongate tubular body 12 to be able to retain a constant diameter when overlying a stricture or lesion without requiring exact placement of the medical stent 10, as placement may not be exact. As an example, in some instances, each of the two or more longitudinally-extending voids 32 may be dimensioned to allow the length L₂ to be no more than 25 percent of the length L₁. For example, in some embodiments, the length L₂ may be in the range of 5 percent to 25 percent of the length L₁, the length L₂ may be in the range of 10 percent to 25 percent of the length L₁, or the length L₂ may be in the range of 15 percent to 25 percent of the length L₁, for instance. In some instances, when there are longitudinally-extending voids 32 disposed within the distal end region 16 and within the proximal end region 14, the total length of both the distally-placed voids 32 and the proximally-placed voids may define the length L₂, and still the length L₂ may be no more than 25 percent of the length L₁ of the entire length of the tubular body 12 of the medical stent 10.

The longitudinally-extending voids 32 may be formed in any suitable manner. In some instances, the elongate tubular body 12 may be formed to include the longitudinally-extending voids 32 as the tubular body 12 is formed. In some instances, the elongate tubular body 12 may be formed, and the longitudinally-extending voids 32 may be subsequently cut into the elongate tubular body 12, removing portions of the elongate tubular body 12. As an example, the longitudinally-extending voids 32 may be laser cut into the elongate tubular body 12 subsequent to forming the tubular body 12 (e.g., subsequent to braiding/weaving the filaments 26 of the tubular body 12). The longitudinally-extending voids 32 may be mechanically cut into the elongate tubular body 12. In some instances, regardless of how the longitudinally-extending voids 32 are formed, a number of welds 36 may be formed around the periphery of each longitudinally-extending void 32 to secure cut ends of the filament(s) or strut(s) 26 together at the periphery of the longitudinally-extending void 32 in order to prevent the elongate tubular body 12 from unraveling or otherwise deforming (absent any applied force) as a result of the cut made into the filaments or struts 26 when forming each of the longitudinally-extending voids 32. In instances in which the tubular body 12 is formed of one or more interwoven filaments, each of the longitudinally-extending dynamic engagement features 34 includes a plurality of filament cross-over points in which either 1) a first filament of the elongate tubular body 12 crosses over a second filament of the elongate tubular body 12, or 2) a first filament segment of a filament of the elongate tubular body 12 crosses over a second filament segment of the filament. Thus, each longitudinally-extending dynamic engagement feature 34 may include a braided or woven filament pattern continuing from, and thus continuous with, the portion of the tubular body 12 proximal of the longitudinally-extending dynamic engagement features 34.

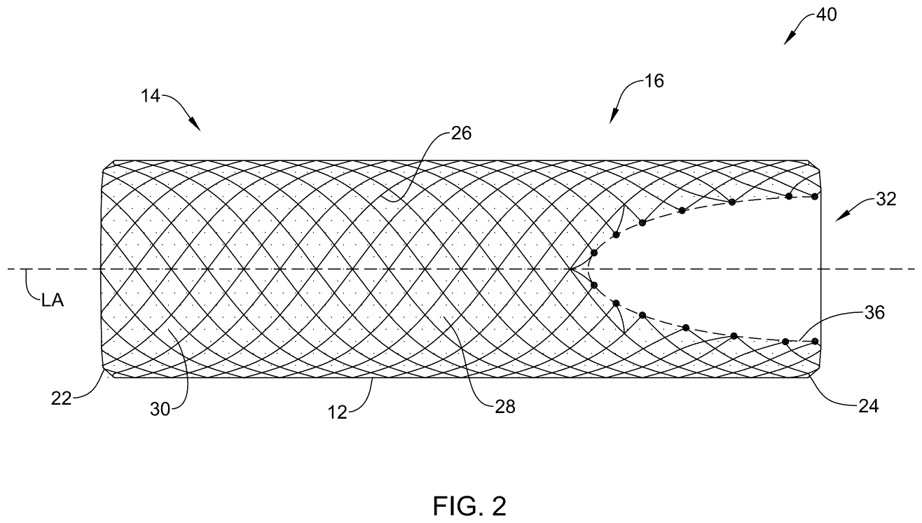

In some instances, as shown for example in FIG. 1, the polymeric coating 30 extends to the distal end 24 of the elongate tubular body 12, and thus extends over or across each of the two or more longitudinally-extending voids 32. In some instances, the polymeric coating 30 may be elastomeric or may otherwise be adapted to stretch in response to radial movement of the two or more longitudinally-extending dynamic engagement features 34 when the two or more longitudinally-extending dynamic engagement features 34 splay radially outwardly in response to an applied axial force. In some instances, the polymeric coating 30 does not extend over the longitudinally-extending voids 32, leaving the longitudinally-extending voids 32 devoid of the polymeric coating 30. FIG. 2 is a schematic view of an example medical stent 40. The example medical stent 40 is quite similar to the medical stent 10, although the polymeric coating 30 does not extend over the longitudinally-extending voids 32, leaving the longitudinally-extending voids 32 devoid of the polymeric coating 30. In some instances, this may permit additional independent radial movement of the longitudinally-extending dynamic engagement features 34 because the longitudinally-extending dynamic engagement features 34 may be unconstrained by any polymeric coating 30 spanning the longitudinally-extending voids 32.

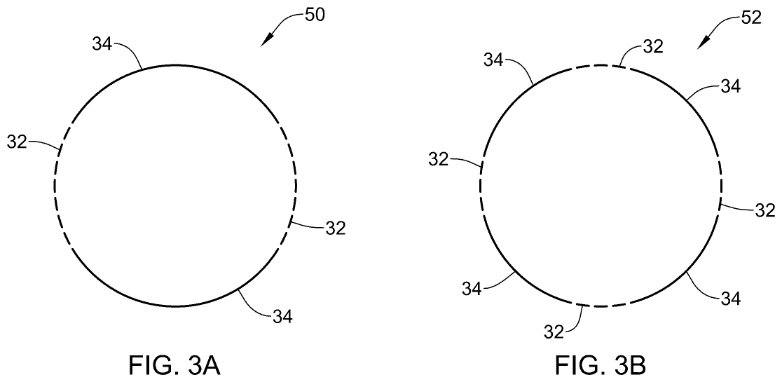

The medical stent 40 (and the medical stent 10) may include any number of longitudinally-extending voids 32 and longitudinally-extending dynamic engagement features 34. FIG. 3A is a schematic end view of an example medical stent 50 that includes a pair of longitudinally-extending voids 32 (i.e., a first longitudinally-extending void 32 and a second longitudinally-extending void 32) and a pair of longitudinally-extending dynamic engagement features 34 (i.e., a first longitudinally-extending dynamic engagement feature 34 and a second longitudinally-extending dynamic engagement feature 34). Each of the longitudinally-extending voids 32 are represented as a dashed line and each of the longitudinally-extending dynamic engagement features 34 are represented as a solid line. The longitudinally-extending voids 32 may be alternatively arranged with the longitudinally-extending dynamic engagement features 34 around the circumference of the tubular body 12. In other words, a longitudinally-extending void 32 may be circumferentially located between adjacent ones of the longitudinally-extending dynamic engagement features 34 around the circumference of the tubular body 12 at the distal end. In the embodiment of FIG. 3A, which includes two longitudinally-extending voids 32 and two longitudinally-extending dynamic engagement features 34, each of the longitudinally-extending voids 32 are circumferentially spaced apart such that each of the longitudinally-extending voids 32 are centered about 180 degrees apart, and each of the longitudinally-extending dynamic engagement features 34 are centered about 180 degrees apart. In some instances, the medical stent 50 may not be symmetric, and the spacing between the longitudinally-extending voids 32 and/or the longitudinally-extending dynamic engagement features 34 may vary from what is shown in FIG. 3A. As there are a total two longitudinally-extending voids 32 and two longitudinally-extending dynamic engagement features 34, the medical stent 50 may be considered as an example of the medical stent 10 (or the medical stent 40).

FIG. 3B is a schematic end view of an example medical stent 52 that includes a total of four longitudinally-extending voids 32 and a total of four of longitudinally-extending dynamic engagement features 34. Each of the longitudinally-extending voids 32 are represented as a dashed line and each of the longitudinally-extending dynamic engagement features 34 are represented as a solid line. In the embodiment of FIG. 3B, each of the longitudinally-extending voids 32 are circumferentially spaced apart such that each of the longitudinally-extending voids 32 are centered about 90 degrees apart, and each of the longitudinally-extending dynamic engagement features 34 are centered about 90 degrees apart. In some instances, the medical stent 52 may not be symmetric, and the spacing between the longitudinally-extending voids 32 and/or the longitudinally-extending dynamic engagement features 34 may vary from what is shown in FIG. 3B. In some instances, the medical stent 52 may include any number of longitudinally-extending voids 32 and longitudinally-extending dynamic engagement features 34, such as three longitudinally-extending voids 32 and longitudinally-extending dynamic engagement features 34, or five longitudinally-extending voids 32 and longitudinally-extending dynamic engagement features 34, or six longitudinally-extending voids 32 and longitudinally-extending dynamic engagement features 34, for example.

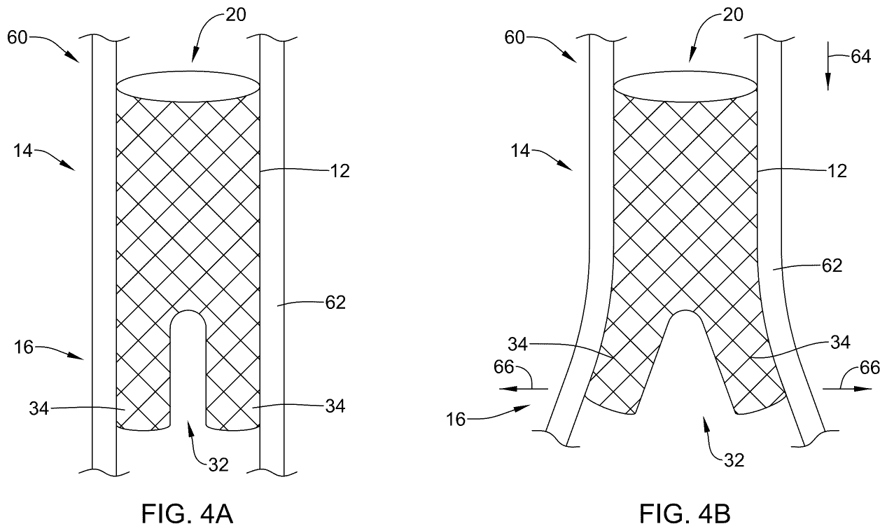

FIGS. 4A and 4B are schematic examples of an example medical stent 60. The medical stent 60 may represent any of the medical stent 10, the medical stent 40, the medical stent 50 or the medical stent 52, described above. The medical stent 60 includes the elongate tubular body 12 and a pair (as shown) of longitudinally-extending voids 32 and a longitudinally-extending dynamic engagement feature 34 disposed on either side of the two longitudinally-extending voids 32. In FIG. 4A, the medical stent 60 may be considered as being in a relaxed state, and may be disposed within a body lumen 62. In FIG. 4B, an applied force is being applied to the medical stent 60 in a direction indicated by an arrow 64. The applied force may be a result of peristaltic motion within the body lumen 62, for example. As the force is applied to the medical stent 60, the medical stent 60 has a tendency to move in the same direction as indicated by the arrow 64. However, as the medical stent 60 is pushed by the applied force, the distal end region 16 is adapted to splay outwards. In other words, as the applied force propagates along the length of the medical stent 60 from the proximal end toward the distal end, the distal end region 16, which includes the longitudinally-extending dynamic engagement features 34, is able to absorb the axial force as the longitudinally-extending dynamic engagement features 34 splay radially outward. As can be seen, the longitudinally-extending dynamic engagement features 34 have a tendency to move radially outwardly in a direction indicated by arrows 66. This helps to anchor the medical stent 60 in position within the body lumen 62 and counteract the axially applied force, such as the force from peristaltic motion of the body lumen 62. As the applied force subsides, the medical stent 60 will relax back into its relaxed state, as seen in FIG. 4A, without appreciably migrating within the body lumen 62.

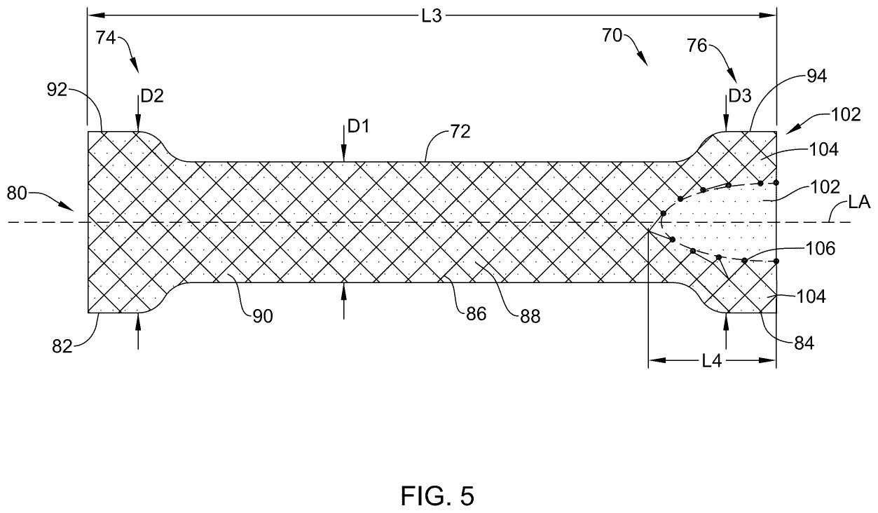

As shown, the medical stent 10, the medical stent 40, the medical stent 50, the medical stent 52 and the medical stent 60 include a cylindrical or largely cylindrical elongate tubular body 12. In some instances, the elongate tubular body 12 has a constant diameter, apart from when applied forces are causing the longitudinally-extending dynamic engagement features 34 to splay in a radially outward direction. In some instances, a medical device (e.g., a medical device) may include one or more flared ends that help to locate and hold the medical device in position within a body lumen. FIG. 5 is a schematic view of an example medical stent 70. The example medical stent 70 includes an elongate tubular body 72. While the medical stent 70 is described as generally tubular, apart from proximal and/or distal flares, it is contemplated that the medical stent 70 may take any cross-sectional shape desired. The elongate tubular body 72 may be considered as including a proximal end region 74, a distal end region 76, and an intervening intermediate region 78, recognizing that these designations are arbitrary, and depend on the orientation in which the medical stent 70 will ultimately be implanted within a body lumen.

The medical stent 70 may include a lumen 80 that extends from a proximal end 82 of the elongate tubular body 72 to a distal end 84 of the elongate tubular body 72 to allow for the passage of food, fluids, etc. The medical stent 70 may be considered as having a longitudinal axis LA extending axially through the lumen 80. The medical stent 70 may be expandable from a first radially collapsed configuration (not shown) to a second radially expanded configuration, which may correspond to its relaxed or equilibrium state. In some instances, the medical stent 70 may be structured to extend across a stricture and to apply a radially outward pressure to the stricture in a body lumen in order to open the body lumen and allow for the passage of foods, fluids, air, etc. therethrough.

In some instances, the medical stent 70 may have a woven structure, fabricated from a number of filaments or struts 86 that together form the elongate tubular body 72. In some instances, the medical stent 70 may be knitted or braided with a single filament or strut interwoven with itself and defining open cells 88 extending through a wall forming the elongate tubular body 72. In some instances, the medical stent 70 may be braided with several filaments or struts interwoven together and defining open cells 88 extending along a length and around the circumference of the tubular wall of the medical stent 70. The open cells 88 may each define an opening from an outer surface of the tubular wall to an inner surface of the tubular wall (e.g., through a thickness thereof) that is free from the filaments or struts 86. Some exemplary stents including braided filaments include the WallFlex®, WALLSTENT®, and Polyflex® stents, made and distributed by Boston Scientific, Corporation. In another embodiment, the medical stent 70 may be knitted, such as the Ultraflex™ stents made by Boston Scientific, Corporation. In yet another embodiment, the medical stent 10 may be of a knotted type, such the Precision Colonic™ stents made by Boston Scientific, Corporation. In still another embodiment, the medical stent 70 may be a monolithic tubular member, for example a laser cut tubular member, such as the EPIC™ stents made by Boston Scientific, Corporation. A laser cut tubular member may have an open and/or closed cell geometry including one or more interconnected monolithic filaments or struts defining open cells 88 therebetween, with the open cells 88 extending along a length and around the circumference of the tubular wall. The open cells 88 may each define an opening from an outer surface of the tubular wall to an inner surface of the tubular wall (e.g., through a thickness thereof) that is free from the interconnected monolithic filaments or struts. In some instances, an inner and/or outer surface of the elongate tubular body 72 may be entirely, substantially, or partially, covered with a polymeric coating 90 that is illustrated as a dotted pattern over the elongated tubular member 72. The polymeric coating 90 may extend across and/or occlude one or more, or a plurality of the open cells 88 defined by the struts or filaments 86. The polymeric coating 90 may help reduce food impaction and/or tumor or tissue ingrowth. In some cases, the medical stent 70 may be a self-expanding stent (SES), configured to automatically radially expand when unconstrained, although this is not required.

In some instances, the elongate tubular body 72 may include one or more of a proximal flare 92 that is positioned within the proximal end region 74 of the elongate tubular body 72 and/or a distal flare 94 that is positioned within the distal end region 76 of the elongate tubular body 72. The proximal flare 92, if included, and the distal flare 94, if included, may be considered as providing improved retention for retaining the medical stent 70 within a particular body lumen. The proximal flare 92 and the distal flare 94 may each be adapted to engage an interior portion of the walls of the esophagus or other body lumen, for example. In some instances, the proximal flare 92 and the distal flare 94 may each have a larger diameter than that of the intermediate region 78 of the elongate tubular body 72 to help prevent the medical stent 70 from migrating once placed in the esophagus or other body lumen. In some instances, a transition from the intermediate region 78 of the elongate tubular body 72 into the proximal flare 92 may be gradual, sloped, or may occur in an abrupt step-wise manner, as desired. In some instances, a transition from the intermediate region 78 of the elongate tubular body 72 into the distal flare 94 may be gradual, sloped, or may occur in an abrupt step-wise manner, as desired.

In some instances, the intermediate region 78 of the elongate tubular body 72 may have a diameter D₁. The proximal flare 92 may have a diameter D₂ that is greater than the diameter D₁. The distal flare 94 may have a diameter D₃ that is greater than the diameter D₁. In some instances, the diameter D₂ may be equal to the diameter D₃. In some instances, the diameter D₂ may be greater than the diameter D₃. In some instances, the diameter D₂ may be less than the diameter D₃. Illustrative examples for the diameter D₁ range from about 6 millimeters to about 30 millimeters. Illustrative examples for the diameter D₂ range from about 12 millimeters to about 45 millimeters. Illustrative examples for the diameter D₃ range from 12 millimeters to about 45 millimeters. In some instances, the medical stent 72 may only include one of the proximal flare 92 and the distal flare 94. For example, the diameter D₂ may be equal to the diameter D₁ (i.e., no proximal flare 92) while the diameter D₃ may be greater than the diameter D₁. In this scenario, the split feature at the distal end may allow for external fluid to flow around the medical stent 70 and not be trapped by the elongate tubular body 72. Alternatively, the diameter D₃ may be equal to the diameter D₁ (i.e., no distal flare 94).

In some instances, the elongate tubular body 72 of the medical stent 70 may be made from a number of different materials such as, but not limited to, metals, metal alloys, shape memory alloys and/or polymers, as desired, enabling the medical stent 70 to be expanded into shape when accurately positioned within the body. In some instances, the material may be selected to enable the medical stent 70 to be removed with relative ease as well. For example, the elongate tubular body 72 of the medical stent 70 can be formed from alloys such as, but not limited to, nitinol and Elgiloy®. Depending on the material selected for construction, the medical stent 70 may be self-expanding or require an external force to expand the medical stent 70. In some embodiments, composite filaments may be used to make the medical stent 70, which may include, for example, an outer shell or cladding made of nitinol and a core formed of platinum or other radiopaque material. It is further contemplated the elongate tubular body 72 of the medical stent 70 may be formed from polymers including, but not limited to, polyethylene terephthalate (PET). In some instances, the filaments of the medical stent 70, or portions thereof, may be bioabsorbable or biodegradable, while in other instances the filaments of the medical stent 70, or portions thereof, may be biostable.

In some instances, the medical stent 70 may be adapted to resist applied forces such as but not limited to those caused by peristaltic motion. In some instances, part of the medical stent 70, such as the distal end region 76 of the medical stent 70, may be adapted to resist motion that would otherwise tend to cause the medical stent 70 to migrate in response to an applied force such as that applied during peristaltic movement. In some instances, the distal end region 76 may include a plurality of longitudinally-extending voids 102, such as two or more longitudinally-extending voids 102, although only a single longitudinally-extending void 102 is visible in this view. Each of the two or more longitudinally-extending voids 102 may be considered as extending from a terminal end (e.g., the distal end 84) in a direction parallel to, or at least substantially parallel to, the longitudinal axis LA. Substantially parallel may be defined as within ten percent of parallel, for example. In some instances, while not shown, one or more of the longitudinally-extending voids 102 may not be parallel or substantially parallel to the longitudinal axis LA, but may instead be disposed at an acute angle with respect to the longitudinal axis LA. In some instances, the distal end region 76 may include a second longitudinally-extending void 102 that is circumferentially spaced about 180 degrees about the elongate tubular body 72. The distal end region 76 may include two, three, four or more longitudinally-extending voids 102, for example.

The longitudinally-extending voids 102 may open out to the distal extent of the medical stent 70 at the distal circumferential edge of the tubular body 72. In some instances, the two or more longitudinally-extending voids 102 may be considered as dividing the distal end region 76 into two or more longitudinally-extending dynamic engagement features 104, e.g., longitudinally-extending legs defined by portions of the wall of the tubular body 72. The two or more longitudinally-extending voids 102 may be considered as defining the longitudinally-extending dynamic engagement features 104 between circumferentially adjacent longitudinally-extending voids 102. In some instances, the two or more longitudinally-extending dynamic engagement features 104 may be considered as being adapted to independently splay radially outwardly in response to an applied peristaltic force, thereby dissipating the applied peristaltic force. In some instances, each of the two or more longitudinally-extending voids 102 extend distally to the distal end 84 of the elongate tubular body 72, such that the distal circumferential edge of the medical stent 70 includes discontinuous segments of the tubular body 12. In some instances, each of the two or more longitudinally-extending voids 102 have the same longitudinal length. In some instances, at least some of the two or more longitudinally-extending voids 102 may have a different length, resulting in varying lengths for the longitudinally-extending dynamic engagement features 104 that are defined at least in part by the two or more longitudinally-extending voids 102.

In some instances, the medical stent 70 may include any number of longitudinally-extending voids 102 and any number of longitudinally-extending dynamic engagement features 104. For example, the medical stent 70 may include two longitudinally-extending voids 102 that are circumferentially spaced part such that they are centered about 180 degrees apart, with the corresponding longitudinally-extending dynamic engagement features 104 also circumferentially spaced apart such that they are centered about 180 degrees apart. As another example, the medical stent 70 may include three longitudinally-extending voids 102 that are circumferentially spaced part such that they are centered about 120 degrees apart, with the corresponding longitudinally-extending dynamic engagement features 104 also circumferentially spaced apart such that they are centered about 120 degrees apart. As another example, the medical stent 70 may include four longitudinally-extending voids 102 that are circumferentially spaced part such that they are centered about 90 degrees apart, with the corresponding longitudinally-extending dynamic engagement features 104 also circumferentially spaced apart such that they are centered about 90 degrees apart.

The elongate tubular body 72 may be considered as having an overall length L₃ that may range from 40 millimeters to 200 millimeters for an esophageal application and may range from 40 millimeters to 140 millimeters for a biliary application. These dimensions are exemplary. In some instances, the length L₄ may range from 5 millimeters to 80 millimeters. In some instances, the length L₄ may range from about 5 percent of the length L₃ to about 40 percent of the length L₃. In some instances, there is a desire to center the medical stent 70 on a stricture or treatment area, with equal portions of the medical stent 70 on either side of the stricture or treatment area. In some instances, the length L₄ is selected to permit the elongate tubular body 72 to be able to retain a constant diameter when overlying a stricture or lesion without requiring exact placement of the medical stent 70, as placement may not be exact. As an example, in some instances, each of the two or more longitudinally-extending voids 102 may be dimensioned to allow the length L₄ to be no more than 25 percent of the length L₃. For example, in some embodiments, the length L₄ may be in the range of 5 percent to 25 percent of the length L₃, the length L₄ may be in the range of 10 percent to 25 percent of the length L₃, or the length L₄ may be in the range of 15 percent to 25 percent of the length L₃, for instance. In some instances, when there are longitudinally-extending voids 102 disposed within the distal end region 76 and within the proximal end region 74, the total length of both the distally-placed voids 102 and the proximally-placed voids may define the length L₄, and still the length L₄ may be no more than 25 percent of the length L₃.

The longitudinally-extending voids 102 may be formed in any suitable manner. In some instances, the elongate tubular body 72 may be formed to include the longitudinally-extending voids 102 as the tubular body 72 is formed. In some instances, the elongate tubular body 72 may be formed, and the longitudinally-extending voids 102 may be subsequently cut into the elongate tubular body 72, removing portions of the elongate tubular body 72. As an example, the longitudinally-extending voids 102 may be laser cut into the elongate tubular body 72 subsequent to forming the tubular body 72 (e.g., subsequent to braiding/weaving the filaments 86 of the tubular body 72). The longitudinally-extending voids 102 may be mechanically cut into the elongate tubular body 72. In some instances, regardless of how the longitudinally-extending voids 102 are formed, a number of welds 106 may be formed around the periphery of each longitudinally-extending void 102 to secure cut ends of the filament(s) or strut(s) 86 together at the periphery of the longitudinally-extending void 102 in order to prevent the elongate tubular body 72 from unraveling or otherwise deforming (absent any applied force) as a result of the cut made into the filaments or struts 86 when forming each of the longitudinally-extending voids 102. In instances in which the tubular body 72 is formed of one or more interwoven filaments, each of the longitudinally-extending dynamic engagement features 104 includes a plurality of filament cross-over points in which either 1) a first filament of the elongate tubular body 72 crosses over a second filament of the elongate tubular body 72, or 2) a first filament segment of a filament of the elongate tubular body 72 crosses over a second filament segment of the filament. Thus, each longitudinally-extending dynamic engagement feature 104 may include a braided or woven filament pattern continuing from, and thus continuous with, the portion of the tubular body 72 proximal of the longitudinally-extending dynamic engagement features 104.

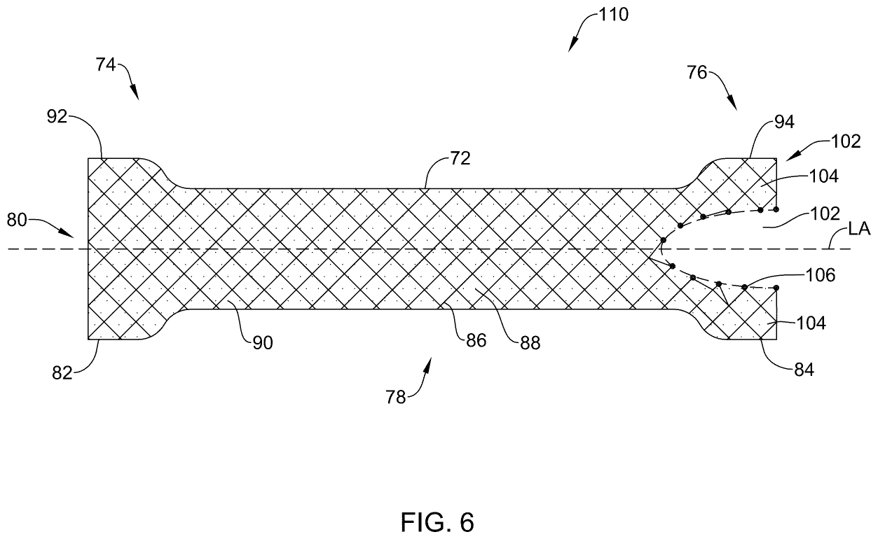

In some instances, as shown for example in FIG. 5, the polymeric coating 90 extends to the distal end 84 of the elongate tubular body 72, and thus extends over or across each of the two or more longitudinally-extending voids 102. In some instances, the polymeric coating 90 may be elastomeric or may otherwise be adapted to stretch in response to radial movement of the two or more longitudinally-extending dynamic engagement features 104 when the two or more longitudinally-extending dynamic engagement features 104 splay radially outwardly in response to an applied axial force. In some instances, the polymeric coating 90 does not extend over the longitudinally-extending voids 102, leaving the longitudinally-extending voids 102 devoid of the polymeric coating 90. FIG. 6 is a schematic view of an example medical device, illustrated as a medical stent 110. The example medical stent 110 is quite similar to the medical stent 70, although the polymeric coating 90 does not extend over the longitudinally-extending voids 102, leaving the longitudinally-extending voids 102 devoid of the polymeric coating 90. In some instances, this may permit additional independent radial movement of the longitudinally-extending dynamic engagement features 104 because the longitudinally-extending dynamic engagement features 104 may be unconstrained by any polymeric coating 90 spanning the longitudinally-extending voids 32.

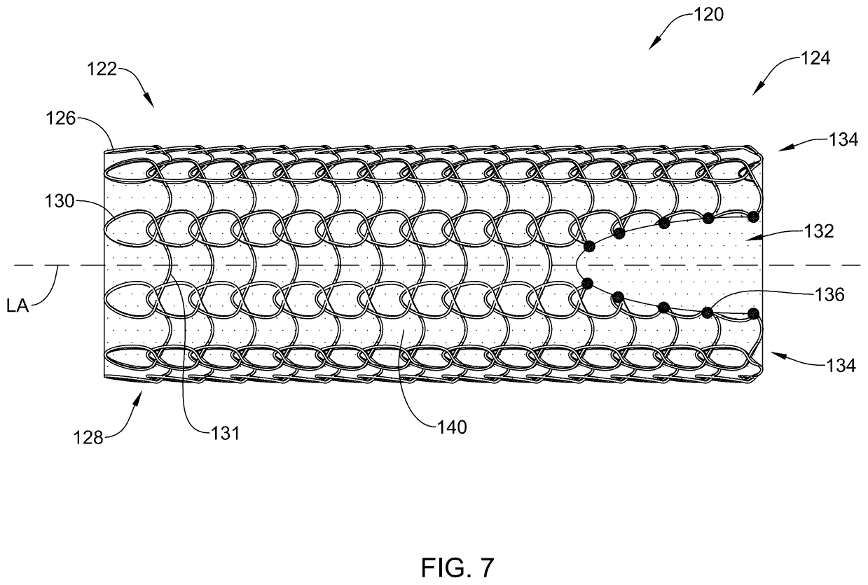

FIG. 7 is a schematic view of an example medical device, illustrated as a medical stent 120 that extends from a proximal end region 122 to a distal end region 124. In some instances, the example medical stent 120 may be an example of a knitted stent, and may be formed from a filament 126 that forms circumferential rows 128 disposed about a longitudinal axis LA of the medical stent 120. In some instances, the medical stent 120 may include a single filament 126, or a plurality of filaments 126. In some instance the medical stent 120 may include one or more interwoven filaments 126. In some instances, the filament 126 may include a number of interwoven filaments. In some instances, the medical stent 120 may have a knitted structure formed and/or fabricated from one or more filaments (or a plurality of filaments) interwoven with each other along the length of the medical stent 120. In some instances, the filament 126 may extend continuously from a first end of the medical stent 120 to a second end of the medical stent 120. In some instances, the circumferential rows 128 may include a plurality of knitted loops 130 and intermediate rungs 131 extending between adjacent knitted loops 130 of and/or within the circumferential rungs 131. At least some of the knitted loops may be closed loops. In some instances, the knitted loops 130 may include open loops or partially open loops. The circumferential rows 128 may extend circumferentially around the longitudinal axis LA.

In some instances, the medical stent 120 may be adapted to resist applied forces such as but not limited to those caused by peristaltic motion. In some instances, part of the medical stent 120, such as the distal end region 124 of the medical stent 120, may be adapted to resist motion that would otherwise tend to cause the medical stent 120 to migrate in response to an applied force such as that applied during peristaltic movement. In some instances, the distal end region 124 may include a plurality of longitudinally-extending voids 132, such as two or more longitudinally-extending voids 132, although only a single longitudinally-extending void 132 is visible in this view. Each of the two or more longitudinally-extending voids 132 may be considered as extending from a terminal end (e.g., the distal end) in a direction parallel to, or at least substantially parallel to, the longitudinal axis LA. Substantially parallel may be defined as within ten percent of parallel, for example. In some instances, while not shown, one or more of the longitudinally-extending voids 132 may not be parallel or substantially parallel to the longitudinal axis LA, but may instead be disposed at an acute angle with respect to the longitudinal axis LA. In some instances, the distal end region 124 may include a second longitudinally-extending void 132 that is circumferentially spaced about 180 degrees from the other longitudinally-extending void 132. The distal region 124 may include two, three, four or more longitudinally-extending voids 132, for example.

The longitudinally-extending voids 132 may open out to the distal extent of the medical stent 120 at the distal circumferential edge of the tubular body of the medical stent 120. In some instances, the two or more longitudinally-extending voids 132 may be considered as dividing the distal end region 124 into two or more longitudinally-extending dynamic engagement features 134, e.g., longitudinally-extending legs defined by portions of the wall of the tubular body of the medical stent 120. The two or more longitudinally-extending voids 132 may be considered as defining the longitudinally-extending dynamic engagement features 134 between circumferentially adjacent longitudinally-extending voids 132. In some instances, the two or more longitudinally-extending dynamic engagement features 134 may be considered as being adapted to independently splay radially outwardly in response to an applied peristaltic force, thereby dissipating the applied peristaltic force. In some instances, each of the two or more longitudinally-extending voids 132 extend distally to a distal end of the tubular body of the medical stent 120, such that the distal circumferential edge of the medical stent 120 includes discontinuous segments of the tubular body. In some instances, each of the two or more longitudinally-extending voids 132 have the same longitudinal length. In some instances, at least some of the two or more longitudinally-extending voids 132 may have a different length, resulting in varying lengths for the longitudinally-extending dynamic engagement features 134 that are defined at least in part by the two or more longitudinally-extending voids 132.

In some instances, the medical stent 120 may include any number of longitudinally-extending voids 132 and any number of longitudinally-extending dynamic engagement features 134. For example, the medical stent 120 may include two longitudinally-extending voids 132 that are circumferentially spaced part such that they are centered about 180 degrees apart, with the corresponding longitudinally-extending dynamic engagement features 134 also circumferentially spaced apart such that they are centered about 180 degrees apart. As another example, the medical stent 120 may include three longitudinally-extending voids 132 that are circumferentially spaced part such that they are centered about 120 degrees apart, with the corresponding longitudinally-extending dynamic engagement features 134 also circumferentially spaced apart such that they are centered about 120 degrees apart. As another example, the medical stent 120 may include four longitudinally-extending voids 132 that are circumferentially spaced part such that they are centered about 90 degrees apart, with the corresponding longitudinally-extending dynamic engagement features 134 also circumferentially spaced apart such that they are centered about 90 degrees apart.

The longitudinally-extending voids 132 may be formed in any suitable manner. In some instances, the medical stent 120 may be formed, and the longitudinally-extending voids 132 may be subsequently cut into the medical stent 120, removing portions of the filament 126. As an example, the longitudinally-extending voids 132 may be laser cut, thereby excising portions of the filament 126. Alternatively, the longitudinally-extending voids 132 may be mechanically cut. In some instances, regardless of how the longitudinally-extending voids 132 are formed, a number of welds 136 may be formed around the periphery of each longitudinally-extending void 132 to secure cut ends of the filament(s) 126 together at the periphery of the longitudinally-extending void 132 in order to prevent the medical stent 120 from unraveling or otherwise deforming (absent any applied force) as a result of the cut made into the filament 126 when forming each of the longitudinally-extending voids 132. In instances in which the tubular body of the medical stent 120 is formed of one or more interwoven filaments, each of the longitudinally-extending dynamic engagement features 134 includes a plurality of filament cross-over points in which either 1) a first filament of the elongate tubular body crosses over a second filament of the elongate tubular body, or 2) a first filament segment of a filament of the elongate tubular body crosses over a second filament segment of the filament. Thus, each longitudinally-extending dynamic engagement feature 134 may include a knitted or woven filament pattern continuing from, and thus continuous with, the portion of the tubular body of the medical stent 120 proximal of the longitudinally-extending dynamic engagement features 134. As shown in FIG. 7, each longitudinally-extending dynamic engagement feature 134 may include a plurality of loops (e.g., open loops or closed loops) of the filament forming the knitted pattern of the tubular body.

In some instances, as shown in FIG. 7, the medical stent 120 may include a polymeric coating 140 that is shown as a dotted pattern. The polymeric coating 140 may cover substantially all of the medical stent 120, including spanning over each of the longitudinally-extending voids 132. In some instances, the polymeric coating 140 may be elastomeric or may otherwise be adapted to stretch in response to radial movement of the two or more longitudinally-extending dynamic engagement features 134 when the two or more longitudinally-extending dynamic engagement features 134 splay radially outwardly in response to an applied axial force. In some instances, as shown, the polymeric coating 140 may not extend over the longitudinally-extending voids 132, leaving the longitudinally-extending voids 132 devoid of the polymeric coating 140.

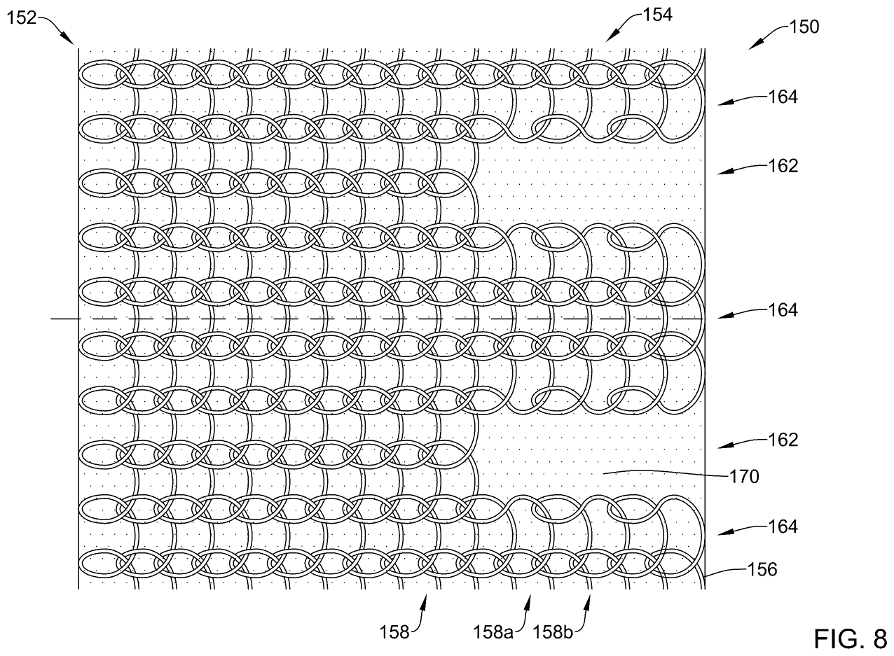

In some instances, instead of cutting the filament 126 to form the longitudinally-extending voids 132, the longitudinally-extending voids 132 may be formed as part of the knitting pattern. FIG. 8 is a schematic view of an example medical stent 150 that extends from a proximal end region 152 to a distal end region 154. In some instances, the example medical stent 150 may be an example of a knitted stent, and may be formed from a filament 156 that shifts from one circumferential row 158A to an adjacent circumferential row 158B at the perimeter of a longitudinally-extending void 162 and reverses circumferential direction around the longitudinal axis LA of the medical stent 150 at the perimeter of a longitudinally-extending void 162. By doing so, this allows for several longitudinally-extending voids 162 to be formed, in combination with several longitudinally-extending dynamic engagement features 164, e.g., longitudinally-extending legs defined by portions of the wall of the tubular body of the medical stent 150. The longitudinally-extending voids 162 may open out to the distal extent of the medical stent 150 at the distal circumferential edge of the tubular body of the medical stent 150.

In instances in which the tubular body of the medical stent 150 is formed of one or more interwoven filaments, each of the longitudinally-extending dynamic engagement features 164 includes a plurality of filament cross-over points in which either 1) a first filament of the elongate tubular body crosses over a second filament of the elongate tubular body, or 2) a first filament segment of a filament of the elongate tubular body crosses over a second filament segment of the filament. Thus, each longitudinally-extending dynamic engagement feature 164 may include a knitted or woven filament pattern continuing from, and thus continuous with, the portion of the tubular body of the medical stent 150 proximal of the longitudinally-extending dynamic engagement features 164. As shown in FIG. 8, each longitudinally-extending dynamic engagement feature 164 may include a plurality of loops (e.g., open loops or closed loops) of the filament forming the knitted pattern of the tubular body.

In some instances, the medical stent 150 may include any number of longitudinally-extending voids 162 and any number of longitudinally-extending dynamic engagement features 164. For example, the medical stent 150 may include two longitudinally-extending voids 162 that are circumferentially spaced part such that they are centered about 180 degrees apart, with the corresponding longitudinally-extending dynamic engagement features 164 also circumferentially spaced apart such that they are centered about 180 degrees apart. As another example, the medical stent 150 may include three longitudinally-extending voids 162 that are circumferentially spaced part such that they are centered about 120 degrees apart, with the corresponding longitudinally-extending dynamic engagement features 164 also circumferentially spaced apart such that they are centered about 120 degrees apart. As another example, the medical stent 150 may include four longitudinally-extending voids 162 that are circumferentially spaced part such that they are centered about 90 degrees apart, with the corresponding longitudinally-extending dynamic engagement features 164 also circumferentially spaced apart such that they are centered about 90 degrees apart.

In some instances, as shown in FIG. 8, the medical stent 150 may include a polymeric coating 170 that is shown as a dotted pattern. The polymeric coating 170 may cover substantially all of the medical stent 150, including spanning over each of the longitudinally-extending voids 162. In some instances, the polymeric coating 170 may be elastomeric or may otherwise be adapted to stretch in response to radial movement of the two or more longitudinally-extending dynamic engagement features 164 when the two or more longitudinally-extending dynamic engagement features 164 splay radially outwardly in response to an applied axial force. In some instances, the polymeric coating 170 may not extend over the longitudinally-extending voids 162, leaving the longitudinally-extending voids 162 devoid of the polymeric coating 170.

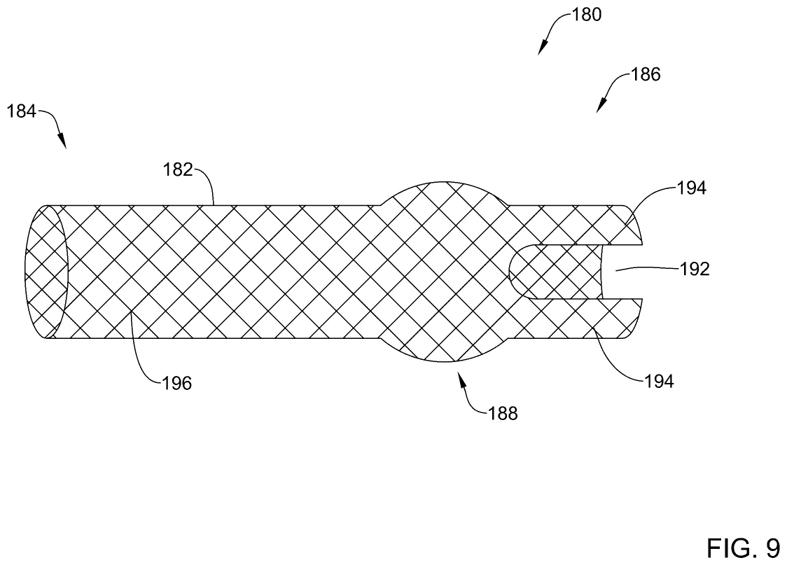

FIG. 9 is a schematic view of an example medical stent 180 including an elongate tubular body 182 extending from a proximal end region 184 to a distal end region 186. In some instances, the elongate tubular body 182 includes a bulbous region 188 along an intermediate region of the tubular body 182 that functions as an anti-migration feature. In some instances, the distal end region 186 of the elongate tubular body 182 may include one or more longitudinally-extending voids 192 that are cut into or otherwise formed within the distal end region 186 of the elongate tubular body 182. This results in longitudinally-extending dynamic engagement features 194, e.g., longitudinally-extending legs, disposed on either side of each of the longitudinally-extending voids 192. A polymeric coating 196 covers substantially all of the medical stent 180, but does not span across the longitudinally-extending voids 192, leaving the longitudinally-extending voids 192 devoid of the polymeric coating 196. The longitudinally-extending voids 192 and longitudinally-extending dynamic engagement features 194 may be of a similar construction, as other embodiments described herein.

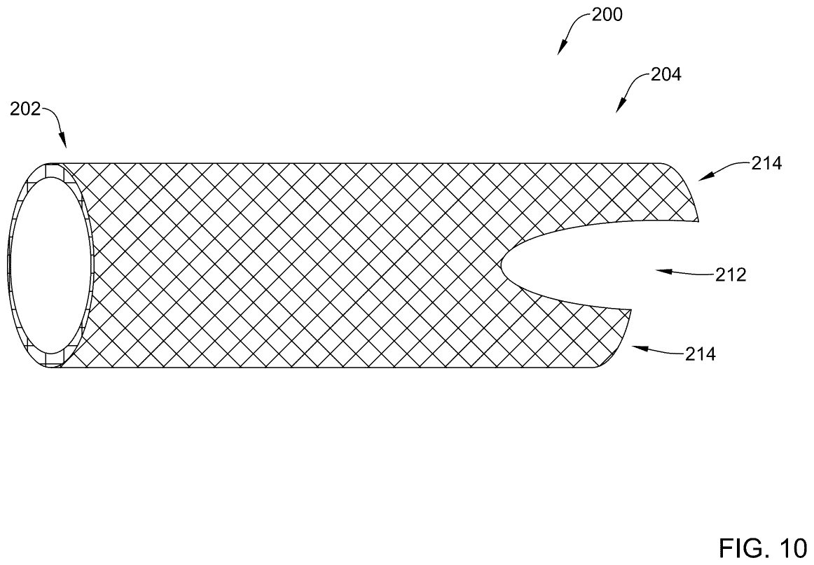

FIG. 10 is a schematic view of an example medical stent 200 that extends from a proximal end region 202 to a distal end region 204. In some instances, the distal end region 204 includes one or more longitudinally-extending voids 212, forming a longitudinally-extending dynamic engagement feature 214, e.g., a longitudinally-extending leg, on either side of the longitudinally-extending void 212. In some instances, one or more of the longitudinally-extending voids 212 may have a different length than one or more of the additional longitudinal-extending voids 212. In some instances, as shown, one or more of the longitudinally-extending dynamic engagement features 214 may have a differing length than one or more of the additional longitudinal-extending dynamic engagement features 214. In some instances, this may contribute to improved place holding during applied forces such as those that result from peristaltic motion.

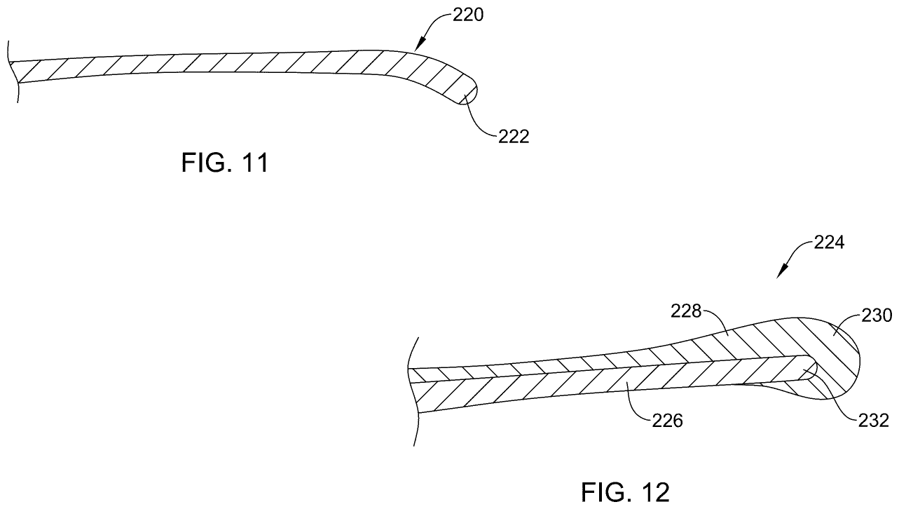

In some instances, the medical devices described herein may have longitudinally-extending dynamic engagement features that are adapted to be atraumatic. FIG. 11 shows a distal part of a longitudinally-extending dynamic engagement feature 220 that may be considered as an example of any of the longitudinally-extending dynamic engagement features described herein. The longitudinally-extending dynamic engagement feature 220 includes a distal tip 222 that is curved away from a plane extending through the rest of the longitudinally-extending dynamic engagement feature 220. In some instances, the distal tip 222 may curve radially inwardly toward the central longitudinal axis of the medical device. In some instances, the distal tip 222 may curve radially outwardly away from the central longitudinal axis of the medical device. FIG. 12 shows a distal part of a longitudinally-extending dynamic engagement feature 224 that includes a structural element 226 and a polymeric coating 228 along at least one side of the structural element 226. In some instances, the polymeric coating 228 may include a thickened portion 230 that wraps around a distal end 232 of the structural element 226 forming a bulbous tip at a distal end of the longitudinally-extending dynamic engagement feature 224. The structural element 226 may represent the elongate tubular body formed from the filament or struts forming the elongate tubular body, for example.

The materials that can be used for the various components of the medical stent(s), the mandrel, and the various elements thereof disclosed herein may include those commonly associated with medical devices and mandrels. For simplicity purposes, the following discussion refers to the apparatus. However, this is not intended to limit the devices and methods described herein, as the discussion may be applied to other elements, members, components, or devices disclosed herein, such as, but not limited to, the medical stent, the mandrel, the filaments, the anti-migration loops, the covering, and/or elements or components thereof.

In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, antiplatelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical stent, comprising:
an elongate tubular body extending from a proximal end region to a distal end region;
the distal end region of the elongate tubular body adapted to resist motion as a result of peristaltic force, the distal end region including:
two or more longitudinally-extending voids; and
two or more longitudinally-extending dynamic engagement features defined between adjacent longitudinally-extending voids;
wherein the two or more longitudinally-extending dynamic engagement features are adapted to splay radially outwardly in response to an applied peristaltic force, thereby dissipating the applied peristaltic force.

2. The medical stent of claim 1, wherein the distal end region extends to a distal end of the elongate tubular body, and the two or more longitudinally-extending voids extend distally to the distal end.

3. The medical stent of claim 1, wherein each of the two or more longitudinally-extending voids have the same length.

4. The medical stent of claim 1, wherein at least some of the two or more longitudinally-extending voids have a different length.

5. The medical stent of claim 1, wherein the medical stent has an overall length, and the length of each of the two or more longitudinally-extending voids is 25 percent or less of the overall length.

6. The medical stent of claim 1, wherein the elongate tubular body has a constant diameter from the proximal end region to the distal end region when the elongate tubular body is in a relaxed state.

7. The medical stent of claim 1, wherein the distal end region of the elongate tubular body has an increased diameter relative to a diameter of the elongate tubular body proximal of the distal end region.

8. The medical stent of claim 1, wherein the proximal end region of the elongate tubular body has an increased diameter relative to a diameter of the elongate tubular body distal of the proximal end region.

9. The medical stent of claim 1, wherein the elongate tubular body comprises a braided stent body.

10. The medical stent of claim 1, wherein the elongate tubular body comprises a knitted stent body.

11. The medical stent of claim 1, further comprising a bulbous region disposed between the distal end region and the proximal end region.

12. The medical stent of claim 1, further comprising a polymeric coating extending over at least a portion of the elongate tubular body.

13. The medical stent of claim 12, wherein the polymeric coating extends from the proximal end region to the distal end region.

14. The medical stent of claim 12, wherein the two or more longitudinally-extending voids are devoid of the polymeric coating.

15. The medical stent of claim 1, wherein the two or more longitudinally-extending dynamic engagement features are adapted to be atraumatic.

16. A medical stent, comprising:
an elongate tubular body extending from a proximal end region to a distal end region;
a polymeric coating extending over at least a portion of the elongate tubular body;
at least one of the proximal end region and the distal end region adapted to resist migration in a body lumen as a result of an applied force, the region configured to resist migration including:
a plurality of longitudinally-extending voids; and
a plurality of longitudinally-extending dynamic engagement features defined between adjacent longitudinally-extending voids;
wherein the plurality of longitudinally-extending dynamic engagement features are adapted to splay radially outwardly in response to the applied force, thereby dissipating the applied force.

17. The medical stent of claim 16, wherein the region configured to resist migration as a result of the applied force comprises the distal end region.

18. The medical stent of claim 16, wherein the region configured to resist migration as a result of the applied force comprises the proximal end region.

19. A medical stent, comprising:
an elongate tubular body extending from a proximal end region to a distal end region;
the elongate tubular body including two or more longitudinally-extending voids extending proximally from a distal end of the elongate tubular body and defining two or more two or more longitudinally-extending dynamic engagement features therebetween;
wherein the two or more longitudinally-extending dynamic engagement features are adapted to independently splay radially outwardly in response to an applied peristaltic force, thereby dissipating the applied peristaltic force.

20. The medical stent of claim 19, wherein each of the two or more longitudinally-extending dynamic engagement features includes a plurality of filament cross-over points in which either 1) a first filament of the elongate tubular body crosses over a second filament of the elongate tubular body, or 2) a first filament segment of a filament of the elongate tubular body crosses over a second filament segment of the filament.
